(19) United States
(12) Patent Application Publication
(10) Pub. No.: US 2014/0208835 A1
(43) Pub. Date: Jul. 31, 2014

(54) METHOD FOR DETERMINATION OF STROKE AND/OR CEREBRAL INFARCTION USING 3-HPMA AS MEASURE

(71) Applicants: Kazuei Igarashi, Chiba-shi (JP); Keiko Kashiwagi, Chiba-shi (JP); Madoka Yoshida, Chiba-shi (JP); Mutsumi Mizoi, Chiba-shi (JP); Ryotaro Saiki, Chiba-shi (JP)

(72) Inventors: Kazuei Igarashi, Chiba-shi (JP); Keiko Kashiwagi, Chiba-shi (JP); Madoka Yoshida, Chiba-shi (JP); Mutsumi Mizoi, Chiba-shi (JP); Ryotaro Saiki, Chiba-shi (JP)

(73) Assignee: Amine Pharma Research Institute Co., Ltd., Chiba (JP)

(21) Appl. No.: 14/123,172

(22) PCT Filed: Jun. 12, 2012
(86) PCT No.: PCT/JP2012/064960

§ 371 Date: Feb. 21, 2014

(30) Foreign Application Priority Data
Jun. 14, 2011 (JP) 2011-132584

(51) Int. Cl. G01N 33/49; G01N 33/493

(52) U.S. Cl. CPC G01N 33/49; G01N 33/493
USPC 73/61.43

(57) ABSTRACT

Provided is a novel assessment method for stroke and cerebral infarction (in particular, asymptomatic cerebral infarction) without any burden on a subject to be tested. It is newly found that the content of 3-HPMA, which is a metabolite of acrolein, in a urine specimen decreases in a stroke patient as compared to a healthy subject without history of stroke, irrespective of smoking habits and gender, which is quite different from the result expected from a conventional finding. Thus, an assessment method for stroke and/or cerebral infarction, including using 3-HPMA in a urine specimen as an indicator, is completed.

### TECHNICAL FIELD

The present invention relates to an assessment method for stroke and/or cerebral infarction, including using 3-hydroxypropyl mercapturic acid (hereinafter referred to as “3-HPMA”) as an indicator.

The present applicant claims priority from Japanese Patent Application No. 2011-132584, which is incorporated herein by reference.

### BACKGROUND ART

### Cerebrovascular Diseases

The number of deaths due to cerebrovascular diseases in Japan ranks third behind those due to malignant neoplasms and heart diseases. Patients with cerebrovascular diseases suffer from aftereffects such as paralysis and akinesia, which not only cause extremely serious problems in daily lives of the patients themselves but also put a great deal of mental stress on caregivers.

Stroke, which accounts for the majority of the cerebrovascular diseases, is a disease that is difficult to early detect and early treat. Although it is effective to initiate therapy at the stage of cerebral infarction (asymptomatic cerebral infarction) without subjective symptoms of cerebral infarction, such as hemiplegia, hemiparesis, numbness, hyposensitivity, limb movement disorder, consciousness disorder, and language disorder, asymptomatic cerebral infarction is casually detected by image diagnosis in most cases.

(Biomarkers for Cerebral Infarction)

Acrolein and a polyamine oxidase, which produces acrolein from a polyamine, are known as biomarkers correlated with cerebral infarction. Acrolein is detoxified by aldehyde dehydrogenase in cells, but exhibits strong toxicity when leaking out of the cells. Thus, acrolein is considered to be strongly correlated with a degree of cell damage, and studies have been made on its correlations with symptoms such as nephropathy, stroke, and asymptomatic cerebral infarction (see: Non Patent Literatures 1 to 3 and Patent Literatures 1 and 2).

(Interleukin-6)

Interleukin-6, which has been discovered as a differentiation inducing factor for B cells, is known to be a malignant cell growth factor for multiple myeloma and to be involved in various inflammatory diseases and autoimmune diseases. Thus, interleukin-6 has been studied as a biomarker for stroke (see: Non Patent Literatures 4 and 5 and Patent Literature 3).

(C-Reactive Protein (CRP))

CRP, which has been discovered as a serum protein (p-globulin) that causes a precipitation reaction with a C-polysaccharide extracted from the cell wall of Streptococcus pneumoniae, is known to show an increased concentration in blood in a number of diseases such as infectious diseases (in particular, bacterial infections), myocardial infarction, and autoimmune diseases.

In recent years, along with a technical improvement of an measuring instrument, a measurement method for high sensitivity CRP (hs-CRP) has been developed, which can sense an extremely mild inflammatory reaction in the absence of apparent inflammatory diseases such as infectious diseases and malignant tumors (minimum detection sensitivity: 0.01 mg/L).

Large clinical studies of Ridker et al. reported that CRP served as an independent prediction marker for ischemic heart disease. Thus, CRP has been studied as a biomarker for stroke (see: Non Patent Literatures 6, 7, and 8 and Patent Literature 3).

(Related Patent Literatures)

The related patent literatures disclose the following.

In Patent Literature 1, there is a disclosure of “a diagnosis method for stroke and asymptomatic cerebral infarction, including using, as indicators, contents of a polyamine and acrolein, or an activity of a polyamine oxidase or an amount of a protein in the polyamine oxidase.”

However, the literature neither discloses nor suggests “an assessment method for stroke and cerebral infarction, including using 3-HPMA as an indicator.”

In Patent Literature 2, there is a disclosure of “a detection method for stroke or asymptomatic cerebral infarction, including measuring contents of an aldehyde compound to be produced from a polyamine, interleukin-6, and C-reactive protein, and an activity of a polyamine oxidase or an amount of a protein in the polyamine oxidase, in a biological sample of a subject to be tested (hereinafter sometimes referred to as subject), and using, as indicators, the resultant measured values and age of the subject.”

However, the literature neither discloses nor suggests “an assessment method for stroke and cerebral infarction, including using 3-HPMA as an indicator.”

In Patent Literature 3, there is a disclosure of “an analysis method for presence or amounts of markers in a panel including one or more specific markers for cerebral damage and one or more non-specific markers for cerebral damage.”

However, the literature neither discloses nor suggests “an assessment method for stroke and cerebral infarction, including using 3-HPMA as an indicator.”

### CITATION LIST

### Patent Literature

[PTL 1] JP 2005-304476 A
[PTL 2] WO 2008/142888 A1
[PTL 3] JP 2005-522669 W

### Non Patent Literature

[NPL 1] Sakata, K. et al (2003) Biochem. Biophys. Res. Commun. 305, 143-149
[NPL 2] Tomitori, H. et al (2005) Stroke 36, 2609-2613
[NPL 3] Igarashi, K. et al (2006) Amino Acids 31, 477-483
[NPL 4] Smith, C. J. et al (2004) BMC Neurol. 15, 4:2
[NPL 5] Tzoulaki, I. et al (2006) Circulation. 115, 2119-2127
[NPL 6] Ridker PM. et al (2002) Engl J Med 347, 1557-1565
[NPL 7] Wakugawa, Y. et al (2006) Stroke 37, 27-32
[NPL 8] Tzoulaki, I. et al (2006) Circulation. 115, 2119-2127

### SUMMARY OF INVENTION

### Technical Problem

A detection method for stroke and cerebral infarction that has been put into practical use involves measuring the content of acrolein in blood. However, the method is an invasive assessment method that requires the collection of blood from a subject.

Thus, an object of the present invention is to provide a novel assessment method for stroke and cerebral infarction (in particular, asymptomatic cerebral infarction) without any burden on a subject.

### Solution to Problem

In order to achieve the object, the inventors of the present invention have made studies on correlations between components in urine and stroke. Surprisingly, the inventors have newly found the result that the content of 3-HPMA, which is a metabolite of acrolein, in a urine specimen decreases in a stroke patient as compared to a healthy subject without history of stroke, irrespective of smoking habits and gender. The result is quite different from one expected from a conventional finding.

Thus, the inventors have completed an assessment method for stroke and/or cerebral infarction, including using 3-HPMA in a urine specimen as an indicator.

In addition, it can be estimated from the results of Examples to be described later that the decrease in the content of 3-HPMA, which is a metabolite of acrolein, in a urine specimen is due to a deficiency in glutathione in a tissue. With this estimation, the supply of glutathione is effective for the prevention and treatment of stroke and/or cerebral infarction, and the amelioration of symptoms of the stroke and/or cerebral infarction. As specific means, the object can be achieved by administering a glutathione formulation and/or supplying a gene encoding glutathione to a patient.

That is, the present invention is as described below.

“1. An assessment method for stroke and/or cerebral infarction, including using, as an indicator, that a content of 3-HPMA in a urine specimen obtained from a subject is low as compared to that of a healthy subject.
2. An assessment method for stroke and/or cerebral infarction, including using, as an indicator, that a content of 3-HPMA in a urine specimen obtained from a subject is low as compared to that of a healthy subject, in which the indicator is that the content of the 3-HPMA in the urine specimen obtained from the subject is low as compared to a preset cut off value.
3. An assessment method according to the above-mentioned item 1 or 2, in which the cerebral infarction includes asymptomatic cerebral infarction.
4. An assessment method according to any one of the above-mentioned items 1 to 3, further including using, as an indicator, that a content of acrolein in a blood specimen obtained from the subject is high as compared to that of the healthy subject.
5. An assessment method according to any one of the above-mentioned items 1 to 4, in which a concentration of the 3-HPMA in the urine specimen obtained from the subject is corrected with a concentration of creatine in the urine specimen.
6. An assessment kit for stroke and/or cerebral infarction, including a reagent for measuring a content of 3-HPMA in a urine specimen.
7. An assessment kit according to the above-mentioned item 6, further including a reagent for measuring a content of acrolein in a blood specimen.”

### Advantageous Effects of Invention

The assessment method for stroke and/or cerebral infarction, in particular, asymptomatic cerebral infarction of the present invention puts less burden on a subject. In addition, the assessment method of the present invention is an assessment method with high accuracy as compared to a conventional method involving measuring the content of acrolein in a blood specimen.

### BRIEF DESCRIPTION OF DRAWINGS

FIG. 1 illustrates a production process for 3-HPMA.

FIG. 2 show a correlation between age and the concentration of 3-HPMA in a urine specimen in the presence or absence of smoking (Example 2).

FIG. 3 show a correlation between the presence or absence of history of stroke and the concentration of 3-HPMA in a urine specimen (Example 3).

FIG. 4 show a correlation between a gender difference and the concentration of 3-HPMA (Example 4).

FIG. 5 show a correlation between the size of a focal site and the concentration of 3-HPMA in a urine specimen (Example 6).

### DESCRIPTION OF EMBODIMENTS

### Assessment Method for Stroke and/or Cerebral Infarction of Present Invention

The present invention relates to an assessment method for stroke and/or cerebral infarction, including using 3-HPMA in a urine specimen as an indicator.

The present invention is characterized by using, as an indicator, that the content of 3-HPMA in a urine specimen of a subject is low as compared to that of a healthy subject.

(Pharmaceutical Composition for Stroke and/or Cerebral Infarction of Present Invention)

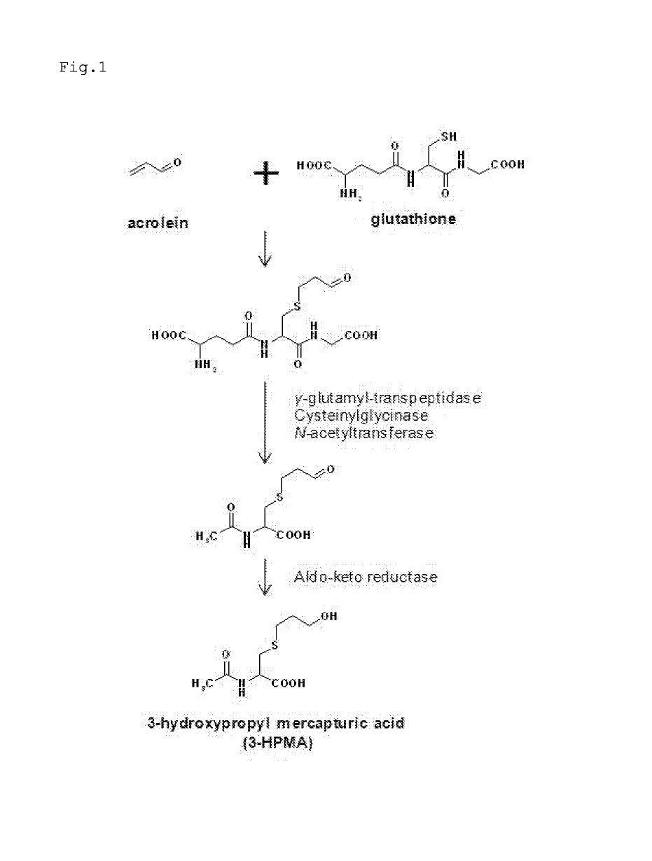

FIG. 1 illustrates the involvement of glutathione in the metabolism of acrolein according to the present invention. Based on the results of Examples to be described later, the inventors of the present invention have estimated that: a deficiency in glutathione, which is a typical compound having an SH group, in a tissue is caused by the promotion of the metabolism of 3-HPMA and subsequently an aldehyde compound by the SH group; and have also estimated that the decrease in the content of 3-HPMA, which is a metabolite of acrolein, in a urine specimen of a stroke patient is based on a decrease in the concentration of glutathione involved in the metabolism of acrolein in a tissue, also due to the age difference or the like in the concentration of glutathione in a tissue.

From the above-mentioned estimations, the concentration of acrolein in a stroke and/or cerebral infarction patient is more decreased by the supply of the SH group (glutathione, which is a typical compound having the SH group) in a tissue. Thus, the supply of the SH group (glutathione, which is a typical compound having the SH group) is effective means for preventing and treating stroke and/or cerebral infarction, and ameliorating symptoms of the stroke and/or cerebral infarction.

The administration of a glutathione formulation and/or the supply of a gene encoding glutathione to a patient allows the concentrations of acrolein in blood and acrolein in a tissue to be decreased to normal ones via the metabolism of acrolein, and moreover, allows the prevention and treatment, and amelioration of the above-mentioned diseases and symptoms to be achieved.

(3-HPMA)

3-Hydroxypropyl mercapturic acid (3-HPMA) is a metabolite of acrolein in urine (see: FIG. 1). In the related technical literatures 1 and 2, there is a disclosure that “the content of acrolein in blood (in particular, plasma) increases in a stroke patient as compared to a subject without history of stroke.”

However, in the present invention, surprisingly, it has been newly found that the content of 3-HPMA, which is a metabolite of acrolein, in urine decreases in a stroke patient as compared to a subject without history of stroke, irrespective of smoking habits and gender.

(Indicator)

The “indicator” of the present invention means a value for the content of 3-HPMA in a urine specimen for distinguishing a stroke and/or cerebral infarction patient (in particular, an asymptomatic cerebral infarction patient) from a healthy subject. For example, when the value for the content of 3-HPMA in a urine specimen of a subject is equal to or lower than a preset value, the development of asymptomatic cerebral infarction and a risk of the development of cerebral infarction in the future and/or the development of stroke in the future can be assessed. Thus, the subject needs to be subjected to head tomography by MRI and/or National Institute of Health Stroke Scale (NIHSS) assessment.

For a setting method for a cut off value, the cut off value is calculated from an average of the contents of 3-HPMA in urine specimens of subjects without history of stroke. In general, in the case of 90% or less, preferably 80% or less, more preferably 70% or less, still more preferably 60% or less, most preferably 50% or less of the standard deviation of a predetermined cut off value, a subject can be assessed as possibly having stroke or asymptomatic cerebral infarction.

Further, as another setting method for a cut off value, based on values obtained by measuring the contents of 3-HPMA in urine specimens of a cerebral infarction patient (in particular, an asymptomatic cerebral infarction patient) and a subject without history of stroke, a receiver operating characteristic (ROC) curve is prepared by using commercially available statistical analysis software to determine an optimum sensitivity and specificity. For example, it is possible to preferentially adopt a cut off value that gives a higher sensitivity for the purpose of, for example, primary screening, and to set a cut value that gives a higher specificity for the purpose of thorough examination.

In addition, based on the results of Examples 2 to 4 below, the cut off value may be set as described below.

(1) The cut off value of a male non-smoker is 1.25 mM to 3.25 mM, preferably 1.25 mM to 2.75 mM, more preferably 1.25 mM to 2.25 mM.
(2) The cut off value of a male smoker is 7.76 mM to 9.76 mM, preferably 7.76 mM to 9.26 mM, more preferably 7.76 mM to 8.76 mM.
(3) The cut off value of a female is 0.76 mM to 1.76 mM, preferably 0.76 mM to 1.51 mM, more preferably 0.76 mM to 1.26 mM.

(Subject)

The subject of the present invention includes mammals including humans. The mammals encompass any animals classified as mammals including humans, domestic animals, non-human primates, animals for athletics (horses for horse racing), or animals for pets, such as dogs, horses, cats, and cattle.

(Measurement Method for 3-HPMA in Urine Specimen)

A method known per se may be utilized as a measurement method for 3-HPMA in a urine specimen (see: Eckert, E. et al. J. Chromatogr. B878, 2506-2514 (2010)).

For example, the content of 3-HPMA in the urine specimen may be measured by using high performance liquid chromatography (HPLC) or LC-MS/MS known per se. It should be noted that the urine specimen means a sample derived from urine, and includes untreated urine, urine supplemented with chemicals, and purified urine.

The content may be preferably measured by enzyme-linked immunosorbent assay (ELISA), western blotting analysis, immunoprecipitation, or the like using an antibody specific for 3-HPMA. The antibody against 3-HPMA to be used in the measurement may be a monoclonal antibody or a polyclonal antibody.

In addition, the concentration of 3-HPMA in a urine specimen is preferably corrected with the concentration of creatine in the urine specimen.

The polyclonal antibody against 3-HPMA may be obtained by, for example, immunizing a rabbit with 3-HPMA using a general technique for producing a peptide antibody.

The production of the antibody may be confirmed by measuring an antibody titer of blood collected from a rabbit to which a peptide has been administered, and testing whether or not the antibody titer reaches a sufficient one.

(Polyamine)

The “polyamine” of the present invention means a linear aliphatic hydrocarbon having two or more primary amino groups. As a known biogenic polyamine, there are given putrescine, cadaverine, spermidine, spermine, 1, 3diaminopropane, caldine, homospermidine, 3-aminopropylcadaverine, norspermine, thermospermine, caldopentamine, and the like, but the polyamine is by no means limited thereto. It should be noted that of those, putrescine, spermidine, or spermine is suitably used as the polyamine in the present invention.

The polyamine is metabolized through oxidation, acetylation, amino group transfer, or carbamoylation, and a polyamine oxidase (acetylpolyamine oxidase (AcPAO) or spermine oxidase (SMO)) is an enzyme involved in the oxidation of the polyamine. It should be noted that herein, the polyamine oxidase means an enzyme generating hydrogen peroxide through the oxidation of a diamine or a polyamine as a suitable substrate. The polyamine undergoes oxidative deamination by a polyamine oxidase, resulting in the production of an aldehyde compound such as acrolein. It should be noted that the aldehyde compound suitable in the present invention is acrolein, but is by no means limited thereto.

(Measurement Method for Acrolein)

The content of acrolein in a blood specimen, in particular, plasma may be identified by any method known to a person skilled in the art, for example, measuring the content of FDP-lysine (N-formylpiperidinolysine), which is an acrolein-amino acid adduct. The content of FDP-lysine may be measured by, for example, using ACR-LYSINE ADDUCT ELISA SYSTEM (NOF CORPORATION) according to the accompanying manual. It should be noted that the content of acrolein may also be measured in a form of a derivative other than FDP-lysine. Alternatively, the content of acrolein may be directly measured, and such method is disclosed in, for example, the report of Alarcon et al. (Alarcon, R. A. (1968) Anal. Chem. 40, 1704-1708).

Specifically, plasma derived from a subject and a standard solution are dispensed into the wells of an antigen-immobilized plate at 50 μL/well, and the same amount of a primary reaction antibody solution is further added. The plate is left to stand still at room temperature for 30 minutes. After that, the solution is removed, the wells are washed with a washing solution, and then a secondary reaction antibody solution is dispensed into the wells at 100 μL/well. The plate is left to stand still at room temperature for 1 hour. After that, the wells are washed with a washing solution, and a color development solution is added to the wells at 100 μL/well. The plate is left to standstill at room temperature for 15 minutes to perform color development, a reaction stop solution is dispensed into the wells at 50 μL/well, and then an absorbance at 450 nm is measured with a plate reader. The amount of acrolein in plasma is expressed as the content of FDP-lysine per mL of plasma (nmol/mL plasma).

(Measurement Method for Interleukin-6)

The content of interleukin-6 in plasma may be measured by any method known to a person skilled in the art, for example, using Human IL-6 ELISA (ENDOGEN) according to the accompanying manual.

Specifically, a primary reaction antibody solution is dispensed into the wells of a 98-well plate at 50 μL/well, and the same amounts of subject plasma and a standard solution are further added. The plate is left to stand still at room temperature for 2 hours. After that, the solution is removed, the wells are washed with a washing solution, and then a secondary reaction antibody solution is dispensed into the wells at 100 μL/well. The plate is left to stand still at room temperature for 30 minutes, and then the wells are washed with a washing solution. After that, a color development solution is added to the wells at 100 μL/well, the plate is left to stand still at room temperature for 30 minutes to perform color development, a reaction stop solution is dispensed into the wells at 100 μL/well, and then an absorbance at 450 nm is measured with a plate reader. The amount of interleukin-6 in plasma is expressed as the content per ml of plasma (pg/mL plasma).

(Measurement Method for CRP)

The content of CRP in plasma may be measured by any method known to a person skilled in the art, for example, using Human CRP ELISA KIT (Alpha Diagnostics) according to the accompanying manual.

Specifically, subject plasma and a standard solution are dispensed into the wells of a 98-well plate at 10 μL/well after the wells have been washed with a washing solution, and an antibody enzyme labeling solution is further added to the wells at 100 μL/well The plate is left to stand still at room temperature for 30 minutes. After that, the solution is removed, and the wells are washed with a washing solution. A color development solution is added, the plate is subjected to color development while being shaken at room temperature for 10 minutes, a reaction stop solution is dispensed into the wells at 50 μL/well, and then an absorbance at 450 nm is measured with a plate reader. The amount of CRP in plasma is expressed as CRP per ml of patient plasma (mg/dL plasma).

(Measurement Method for Polyamine Oxidases)

The activities of polyamine oxidases (AcPAO and SMO) may be measured by any method known to a person skilled in the art, for example, incubating 0.06 ml of a reaction mixed solution of 10 mM Tris-hydrochloride (pH 7.5), a 0.2 mM substrate (acetyl spermine or spermine), and 0.05 ml of patient plasma at 37° C. for 48 hours. To 0.02 ml of the reaction mixed solution is added trichloroacetic acid (TCA) at a final concentration of 5%, and the mixture is centrifuged. Part of the resultant supernatant is used in an assay for polyamines. The activities of the polyamine oxidases may be expressed as the amounts of spermidine produced by the decomposition of acetyl spermine or spermine per ml of patient plasma (nmol/ml plasma).

The measurement methods for the enzymatic activities of the polyamine oxidases are disclosed in various reports, and as such documents, there may be given a report of Sharmin et al. (Sharmin et al., (2001) Biochem. Biophys. Res. Commun. 282, 228-235), a report of Sakata et al. (Sakata et al., (2003) Biochem. Biophys. Res. Commun. 305, 143-149), a report of Igarashi et al. (Igarashi et al., (1986) J. Bacteriol. 166, 128-134), and the like. The enzymatic activities of the polyamine oxidases may be measured by a person skilled in the art based on the disclosures of such reports by appropriately modifying the disclosures.

Further, the amounts of proteins in the polyamine oxidases may be measured any method known to a person skilled in the art, such as enzyme-linked immunosorbent assay (ELISA), western blotting analysis, immunoprecipitation, or the like using antibodies specific for the polyamine oxidases. Such technique is a general technique known in the art, and a person skilled in the art can appropriately set proper conditions and measure the amounts of proteins in the enzymes by the above-mentioned technique. It should be noted that the antibodies against the polyamine oxidases to be used in the measurement may be a monoclonal antibody or a polyclonal antibody.

(Analysis Method Using Measured Values)

In the present invention, the content of 3-HPMA in a urine specimen, the contents of an aldehyde compound to be produced from a polyamine, interleukin-6, and C-reactive protein, and the activities of polyamine oxidases or the amounts of proteins in the polyamine oxidases in a biological sample are measured, and the resultant measured values and the age of a subject are subjected to mathematical statistical analysis. As a result of the analysis, a value that gives a statistically significant change is obtained. Based on the value, stroke and/or cerebral infarction can be assessed.

The mathematical statistical analysis may be performed by a method known to a person skilled in the art, preferably using a neural network technique. The neural network technique may be performed by, for example, using NEUROSIM/L (FUJITSU LIMITED) according to the accompanying manual.

Further, the value that gives a statistically significant change obtained in the foregoing can be used as a “cut off value” to assess stroke or cerebral infarction. For example, based on values obtained as a result of the measurement of the contents of the biomarkers in an asymptomatic cerebral infarction patient and a healthy subject, a receiver operating characteristic (ROC) curve is prepared by using commercially available statistical analysis software to determine an optimum sensitivity and specificity. Depending on the purposes of the assessment, it is possible to preferentially adopt a cut off value that gives a higher sensitivity for the purpose of, for example, primary screening, and to set a cut value that gives a higher specificity for the purpose of thorough examination.

(Screening Method for Therapeutic Agent or Preventive Agent for Stroke and/or Cerebral Infarction)

The present invention provides a screening (search) method for a novel medicament effective for the treatment and prevention of stroke and/or cerebral infarction, the method including administering a candidate compound that may be effective for the treatment of stroke and/or cerebral infarction to an experimental animal, and measuring whether or not the compound promotes the production of 3-HPMA in the experiment animal.

(Assessment Kit for Stroke and Asymptomatic Cerebral Infarction)

The present invention provides an assessment kit for stroke or asymptomatic cerebral infarction. The kit includes a reagent for measuring the content of 3-HPMA in a urine specimen. The kit further includes, as necessary, a reagent for measuring the content of acrolein in a blood specimen, and reagents for measuring the contents of an aldehyde compound to be produced from a polyamine, interleukin-6, and C-reactive protein, and the activities of polyamine oxidases or the amounts of proteins in the polyamine oxidases. In addition, any measuring instrument or apparatus, standard solution, buffer, or the like known to a person skilled in the art may be incorporated as necessary.

In the following, the present invention is described in detail by way of Examples. However, the scope of the present invention is by no means limited to Examples shown below.

It should be noted that Examples shown below were performed according to the Helsinki Declaration.

### Example 1

### Methods

The details of a measurement method for 3-HPMA, a measurement method for creatine, a statistical analysis method, head tomographic analysis, and an NIHSS evaluation method are as described below.

(Measurement Method for 3-HPMA)

The measurement of 3-HPMA was performed according to the method disclosed in the literature Eckert, E. et al. J. Chromatogr. B878, 2506-2514 (2010). The details are as described below. It should be noted that the collection of a urine specimen was performed according to the procedure approved by the ethics committee of Chiba University and Chiba Central Medical Center.

2 ml of urine collected from a subject were mixed with 2 ml of an ammonium formate buffer (50 mM, pH 2.5) and 0.04 ml of formic acid. Next, the mixture was centrifuged (2,000×g, 5 minutes), and then the supernatant was passed through an SPE column (ISOLUTE ENV+, 100 mg, 3 ml) equilibrated with 6 ml of methanol and 6 ml of formic acid (pH 2.5) in advance. The cartridge after the passage was washed and further eluted with 2.5 ml of 2% formic acid (dissolved with methanol). The eluate was dried and finally dissolved with 1 ml of Solvent A (5 mM ammonium acetate, pH 6.5 in acetonitrile/water (88/12, v/v)). The resultant solution was centrifuged (2,000×g, 10 minutes), and then 0.01 ml of the supernatant was used in LC-MS/MS analysis.

It should be noted that LC separation was performed by hydrophilic interaction liquid chromatography (X Bridge HILIC, 3.5 lam particle size, 2.1 mm×150 mm, Waters, Mass., USA) and a corresponding precolumn (HILIC, 2.1 mm×10 mm). The separation of 3-HPMA was performed by using 5 mM ammonium acetate (mixed solution of 88% acetonitrile and 12% water, flow rate condition: 0.3 ml/min) having a uniform concentration and a pH of 6.5. Fractions eluted between 3 to 9 minutes containing 3-HPMA were injected into a detector of MS (model Sciex API 2000, Applied Biosystems, Langen, Germany). An electrospray needle voltage was set to −4,000 V in a negative ion mode. A turbo heater was kept at 475° C. Nitrogen was used as an atomization gas, a heater gas, and a curtain gas. The pressures of the atomization gas, the heater gas, and the curtain gas were set to 45 psi, 60 psi, and 25 psi, respectively. A collision gas (nitrogen) for an MS/MS mode was set to a flow of three instrument units. For the MS, a multiple reaction monitoring mode (MRM) was used. The retention time of 3-HPMA was 4.8 minutes, and a precursor ion (Q1) and a product ion (Q3) were observed at 220.2 m/z and 91.0 m/z, respectively.

(Subject)

A control group consists of healthy volunteers that live independently without apparent history of stroke or dementia.

A stroke patient group was determined based on the presence of a local infarction detected by MRI or CI. It should be noted that the group does not include a chronic renal failure patient.

(Measurement Method for Creatine)

The measurement of creatine was performed by using a commercially available creatine assay kit (Cayman Chemical Co., USA).

(Statistical Analysis Method)

Statistical calculation was carried out with GraphPad Prism^{(trademark)} Software (GraphPad Software). Values were shown by median±quartile deviation. The groups were compared by using a Wilcoxon rank-sum test. A Spearman's rank-correlation coefficient was used for investigating a statistical correlation relationship between age and 3-HPMA or creatine.

(Head Tomographic Analysis)

All patients underwent T1 and T2-weighted MRI, and some of the patients underwent fluid-attenuated inversion recovery (FLAIR) and computed tomography (CT). In all the cases, the MRI was carried out with a 1.5T-MRI unit (Signa HiSpeed Infinity, GE Medical Systems) at a slice gap of 1 to 2 mm and a thickness of 5 to 8 mm. A standard head coil of receive-transmit birdcage design was used. The maximum size of a focal infarction was measured with a 5- or 10-mm scale attached to each image.

(NIHSS Evaluation Method)

The NIHSS of this example was evaluated based on the disclosure of the literature Lyden, P. D. et al. Stroke 32, 1310-1317 (2001).

### Example 2

### Confirmation of Correlation Between Age and Concentration of 3-HPMA in Urine Specimen in Presence or Absence of Smoking

Whether or not there was a correlation between age and the concentration of 3-HPMA in a urine specimen in the presence or absence of smoking was confirmed. The details are as described below.

For 106 subjects without history of stroke as a control (non-smoker group: 87 subjects, smoker group: 19 subjects), the concentrations of 3-HPMA in urine specimens and the concentrations of creatinine in urine specimens were measured.

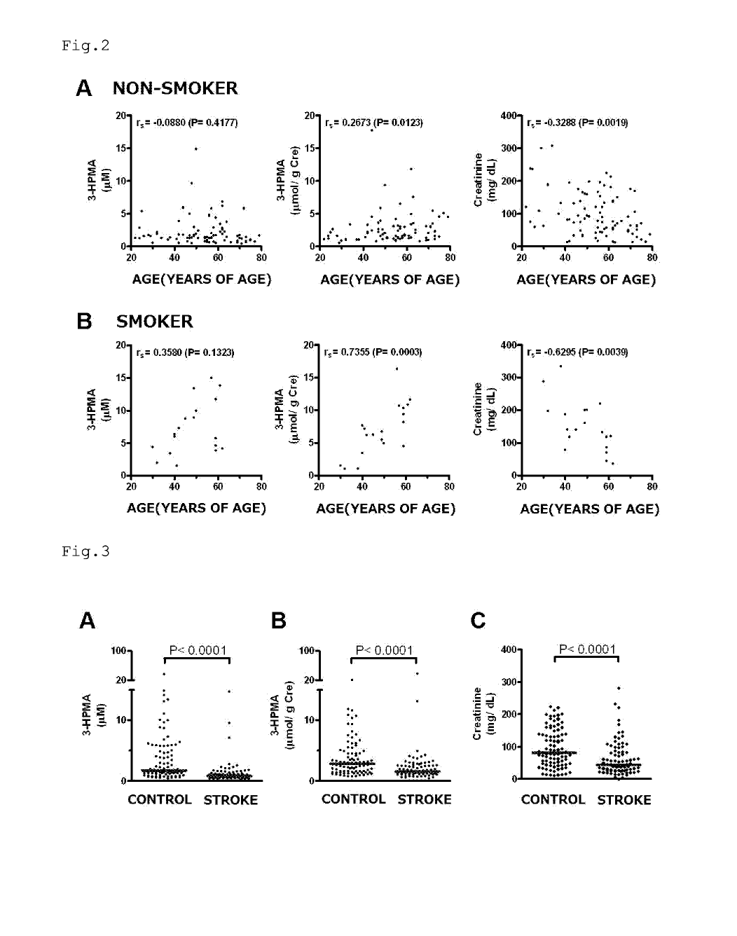

From the measurement results, in the non-smoker group, no correlation with a statistically significant difference was found between the age and the concentration of 3-HPMA in the urine specimen (see: FIG. 2A(a): rs=−0.0880, P=0.4177).

In the smoker group, no correlation with a statistically significant difference was found between the age and the concentration of 3-HPMA in the urine specimen (see: FIG. 2B(a): rs=0.3580, P=0.1323).

In the non-smoker group, a positive correlation with a statistically significant difference was found between the age and the concentration of 3-HPMA in the urine specimen corrected with the concentration of creatinine in the urine specimen (see: FIG. 2A(b): rs=0.2673, P=0.0123).

In the smoker group, a positive correlation with a statistically significant difference was found between the age and the concentration of 3-HPMA in the urine specimen (see: FIG. 2B(b): rs=0.7355, P=0.0003).

Further, in both the non-smoker group and the smoker group, a negative correlation with a statistically significant difference was found between the age and the concentration of creatinine in the urine specimen (FIG. 2A(c): rs=−0.3288, P=0.0019, FIG. 2B(c): rs=−0.6295, P=0.0039).

The above-mentioned results confirmed that the concentration of 3-HPMA in the urine specimen increased along with aging and the concentration of creatinine in the urine specimen decreased along with aging.

### Example 3

### Confirmation of Correlation Between Presence or Absence of History of Stroke and Concentration of 3-HPMA in Urine Specimen

Whether or not there was a correlation between the presence or absence of history of stroke and the concentration of 3-HPMA in a urine specimen was confirmed. The details are as described below.

For a control group consisting of 90 subjects without history of stroke and a stroke patient group consisting of 81 subjects, the concentrations of 3-HPMA in urine specimens and the concentrations of creatinine in urine specimens were measured.

From the measurement results, the concentration of 3-HPMA in the urine specimen was found to decrease with a statistically significant difference in the stroke patient group (median: 0.82 mM) as compared to the control group (median: 1.74 mM) (see: FIG. 3A, P<0.0001).

Further, the concentration of creatinine in the urine specimen was found to decrease with a statistically significant difference in the stroke patient group (median: 44.6 mg/dL) as compared to the control group (median: 81.9 mg/dL) (see: FIG. 3C, P<0.0001).

In addition, the concentration of 3-HPMA in the urine specimen corrected with the concentration of creatinine in the urine specimen was found to decrease with a statistically significant difference in the stroke patient group (median: 1.57 mmol/g Cre) as compared to the control group (median: 2.83 mmol/g Cre) (see: FIG. 3B, P<0.0001).

The above-mentioned results confirmed that the concentration of 3-HPMA in the urine specimen decreased in the stroke patient group as compared to the control group without history of stroke.

### Example 4

### Confirmation of Correlation Between Gender and Concentration of 3-HPMA

Whether or not there was a correlation between gender and the concentration of 3-HPMA was confirmed. The details are as described below.

1. Confirmation of Correlation Between Male Non-Smoker and Concentration of 3-HPMA in Urine Specimen

In male non-smokers, for a control group consisting of 31 subjects without history of stroke and a stroke patient group consisting of 34 subjects, the concentrations of 3-HPMA in urine specimens and the concentrations of creatinine in urine specimens were measured.

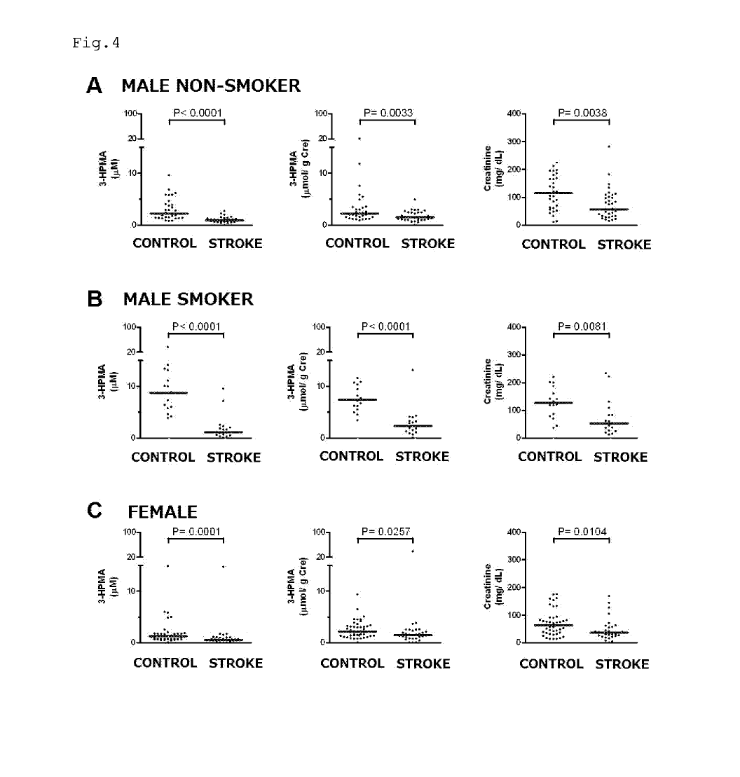

From the measurement results, the concentration of 3-HPMA in the urine specimen was found to decrease with a statistically significant difference in the stroke patient group (median: 0.95 mM) as compared to the control group (median: 2.25 mM) (see: FIG. 4A(a), P<0.0001).

Further, the concentration of creatinine in the urine specimen was found to decrease with a statistically significant difference in the stroke patient group (median: 58.3 mg/dL) as compared to the control group (median: 114.9 mg/dL) (see: FIG. 4A(c), P=0.0038).

In addition, the concentration of 3-HPMA in the urine specimen corrected with the concentration of creatinine in the urine specimen was found to decrease with a statistically significant difference in the stroke patient group (median: 1.56 mmol/g Cre) as compared to the control group (median: 2.31 mmol/g Cre) (see: FIG. 4A(b), P=0.0033).

2. Confirmation of Correlation Between Male Smoker and Concentration of 3-HPMA in Urine Specimen

In male smokers, for a control group consisting of 16 subjects without history of stroke and a stroke patient group consisting of 17 subjects, the concentrations of 3-HPMA in urine specimens and the concentrations of creatinine in urine specimens were measured.

From the measurement results, the concentration of 3-HPMA in the urine specimen was found to decrease with a statistically significant difference in the stroke patient group (median: 1.13 mM) as compared to the control group (median: 8.76 mM) (see: FIG. 4B(a), P<0.0001).

Further, the concentration of creatinine in the urine specimen was found to decrease with a statistically significant difference in the stroke patient group (median: 53.1 mg/dL) as compared to the control group (median: 126.3 mg/dL) (see: FIG. 4B(c), P=0.0081).

In addition, the concentration of 3-HPMA in the urine specimen corrected with the concentration of creatinine in the urine specimen was found to decrease with a statistically significant difference in the stroke patient group (median: 2.96 mmol/g Cre) as compared to the control group (median: 7.40 mmol/g Cre) (see: FIG. 4B(b), P<0.0001).

3. Confirmation of Correlation Between Female and Concentration of 3-HPMA in Urine Specimen

In females, for a control group consisting of 43 subjects without history of stroke and a stroke patient group consisting of 30 subjects, the concentrations of 3-HPMA in urine specimens and the concentrations of creatinine in urine specimens were measured.

From the measurement results, the concentration of 3-HPMA in the urine specimen was found to decrease with a statistically significant difference in the stroke patient group (median: 0.57 mM) as compared to the control group (median: 1.26 mM) (see: FIG. 4C(a), P=0.0001).

Further, the concentration of creatinine in the urine specimen was found to decrease with a statistically significant difference in the stroke patient group (median: 36.2 mg/dL) as compared to the control group (median: 63.2 mg/dL) (see: FIG. 4C(c), P=0.0104).

In addition, the concentration of 3-HPMA in the urine specimen corrected with the concentration of creatinine in the urine specimen was found to decrease with a statistically significant difference in the stroke patient group (median: 1.47 mmol/g Cre) as compared to the control group (median: 2.20 mmol/g Cre) (see: FIG. 4C(b), P<0.0001).

The above-mentioned results first revealed that the concentration of 3-HPMA in the urine specimen decreased in the stroke patient group as compared to the control group without history of stroke, irrespective of smoking habits and gender.

### Example 5

### Confirmation of Correlation Between Population of Same Age Group and Concentration of 3-HPMA in Urine Specimen

Whether or not there was a correlation between a population of the same age group (aged 60 to 79) and the concentration of 3-HPMA was confirmed. The details are as described below.

For a control group consisting of 32 subjects without history of stroke and a stroke patient group consisting of 47 subjects in a population of the same age group (aged 60 to 79), the concentrations of 3-HPMA in urine specimens and the concentrations of creatinine in urine specimens were measured. In addition, the concentrations (mg/dL) of urine urea nitrogen (UUN) were measured.

From the measurement results, the concentration of 3-HPMA in the urine specimen was found to decrease with a statistically significant difference in the stroke patient group as compared to the control group (not shown). On the other hand, no statistically significant difference in the UUN concentration was found in the stroke patient group as compared to the control group (not shown).

### Example 6

### Confirmation of Correlation Between Size of Focal Site and Concentration of 3-HPMA in Urine Specimen

Whether or not there was a correlation between the size of a focal site and the concentration of 3-HPMA in a urine specimen was confirmed. The details are as described below.

For a group in which the size of a focal site of stroke was less than 1 cm consisting of 20 subjects and a group in which the size of a focal site of stroke was 1 cm or more consisting of 56 subjects, the concentrations of 3-HPMA in urine specimens were measured.

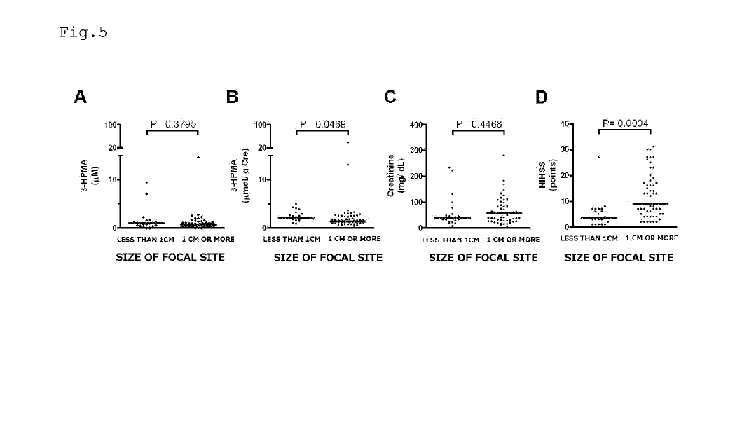

From the measurement results, no statistically significant difference in the concentration of 3-HPMA in the urine specimen was found in the group in which the size of a focal site was 1 cm or more (median: 0.72 mM) as compared to the group in which the size of a focal site was less than 1 cm (median: 1.03 mM) (see: FIG. 5A, P=0.3795).

No statistically significant difference in the concentration of creatinine in the urine specimen was found in the group in which the size of a focal site was 1 cm or more (median: 55.9 mg/dL) as compared to the group in which the size of a focal site was less than 1 cm (median: 38.8 mg/dL) (see: FIG. 5C, P=0.4468).

Further, the concentration of 3-HPMA in the urine specimen corrected with the concentration of creatinine in the urine specimen was found to decrease with a statistically significant difference in the group in which the size of a focal site was 1 cm or more (median: 1.39 mmol/g Cr) as compared to the group in which the size of a focal site was less than 1 cm (median: 2.16 mmol/g Cr) (see: FIG. 5B, P=0.0469).

In addition, no statistically significant difference in the NIHSS, which was a scale for evaluating the severity of stroke, was found in the group in which the size of a focal site was 1 cm or more (median: 9.0 points) as compared to the group in which the size of a focal site was less than 1 cm (median: 3.5 points) (see: FIG. 5D, P=0.0004).

The above-mentioned results confirmed that, as the size of the focal site of stroke became larger, the concentration of 3-HPMA in the urine specimen decreased and the NIHSS showed a higher value.

(General Remarks)

The results of Examples 2 to 6 above revealed that the content of 3-HPMA, which was a metabolite of acrolein, in the urine specimen decreased in the stroke patients as compared to the subjects without history of stroke, irrespective of smoking habits and gender.

In addition, in Example 3, the median of the concentrations of 3-HPMA in the urine specimens of the control group corrected with the concentrations of creatinine in the urine specimens was 2.83 mmol/g Cre, and the median of the concentrations of 3-HPMA in the urine specimens of the stroke patient group corrected with the concentrations of creatinine in the urine specimens was 1.57 mmol/g Cre. In other words, the median of the control group was 1.80-fold as compared to the median of the stroke patient group.

On the other hand, in Non Patent Literature 2, the median of the concentrations of acrolein in blood specimens of a stroke patient group was 1.48-fold as compared to the median of the concentrations of acrolein in blood specimens of a control group.

The above-mentioned results confirmed that the assessment method of the present invention was an assessment method with high accuracy as compared to a conventional method involving measuring the content of acrolein in a blood specimen.

(With Regard to Mechanism of Decrease in Concentration of 3-HPMA in Urine Specimen)

The inventors of the present invention have assumed the mechanism of the decrease in the concentration of 3-HPMA in the urine specimen of the stroke patient, which was found by Examples above, to be as described below.

Acrolein in a tissue is considered to be present in a state of being bound to a protein. The metabolism of acrolein requires glutathione as illustrated in FIG. 1. The fact that the concentration of acrolein in blood is high and the concentration of 3-HPMA, which is a metabolite of acrolein, in urine is low in the stroke patient indicates that the metabolism of acrolein in blood in which glutathione is involved does not proceed for some reasons. On the assumption that acrolein is present in a large amount, this is primarily because only a small amount of glutathione can be involved in the metabolism owing to its low concentration in a tissue, the turnover does not proceed, and the production of 3-HPMA, which is a metabolite, decreases, with the result that the concentration of 3-HPMA in urine decreases.

The dynamic state of the metabolism of acrolein in which glutathione is involved is estimated as described below. Initially, a large amount of glutathione is used for metabolizing a large amount of acrolein into a metabolite, and an SH group exhibits a decomposition promoting action for further decomposing 3-HPMA into an aldehyde compound. As a result, glutathione, which is a typical compound having the SH group, is further consumed. In this case, the deficient state of glutathione in a tissue appears, the metabolism of acrolein in which glutathione is involved is prevented from proceeding, with the result that 3-HPMA, which is a metabolite, is not produced. It is considered that this state continues in the stroke patient, and is manifested as the decrease in the concentration of 3-HPMA in urine.

Further, the concentration of glutathione in a tissue is expected to vary depending on factors such as age.

According to the above-mentioned consideration, in order to additionally decrease acrolein in blood, it is necessary to increase the concentration of glutathione in a tissue to promote the metabolism of acrolein in which glutathione is involved, and supply an SH group to promote the decomposition of 3-HPMA into an aldehyde compound, to thereby more efficiently perform the metabolism of acrolein.

The decrease in acrolein can be achieved by performing, as specific means for supplying glutathione, the administration of a glutathione formulation and/or the supply of a gene encoding glutathione to a patient. Thus, it is considered that the concentrations of acrolein in blood and acrolein in a tissue can be decreased to normal ones via the metabolism of acrolein, and moreover, the prevention and treatment, and amelioration of the above-mentioned diseases and symptoms can be achieved.

### INDUSTRIAL APPLICABILITY

In the present invention, it is possible to provide the assessment method for stroke and/or cerebral infarction, in particular, asymptomatic cerebral infarction, with less burden on a subject, including using 3-HPMA in a urine specimen as an indicator. In addition, the assessment method of the present invention is an assessment method with high accuracy as compared to a conventional method involving measuring the content of acrolein in a blood specimen.

In addition, it is possible to provide an assessment method for stroke and/or cerebral infarction with higher accuracy by adding a conventional measurement result of the content of acrolein in a blood specimen to the assessment method for stroke and/or cerebral infarction, including using 3-HPMA in a urine specimen as an indicator, of the present invention.

1. An assessment method for stroke and/or cerebral infarction, comprising using, as an indicator, that a content of 3-hydroxypropyl mercapturic acid (3-HPMA) in a urine specimen obtained from a subject to be tested is low as compared to that of a healthy subject.

2. An assessment method for stroke and/or cerebral infarction, comprising using, as an indicator, that a content of 3-HPMA in a urine specimen obtained from a subject to be tested is low as compared to that of a healthy subject,
wherein the indicator is that the content of the 3-HPMA in the urine specimen obtained from the subject to be tested is low as compared to a preset cut off value.

3. An assessment method according to claim 1, wherein the cerebral infarction comprises asymptomatic cerebral infarction.

4. An assessment method according to claim 1, further comprising using, as an indicator, that a content of acrolein in a blood specimen obtained from the subject to be tested is high as compared to that of the healthy subject.

5. An assessment method according to claim 1, wherein a concentration of the 3-HPMA in the urine specimen obtained from the subject to be tested is corrected with a concentration of creatine in the urine specimen.

6. An assessment kit for stroke and/or cerebral infarction, comprising a reagent for measuring a content of 3-HPMA in a urine specimen.

7. An assessment kit according to claim 6, further comprising a reagent for measuring a content of acrolein in a blood specimen.

8. An assessment method according to claim 3, further comprising using, as an indicator, that a content of acrolein in a blood specimen obtained from the subject to be tested is high as compared to that of the healthy subject.

9. An assessment method according to claim 3, wherein a concentration of the 3-HPMA in the urine specimen obtained from the subject to be tested is corrected with a concentration of creatine in the urine specimen.

10. An assessment method according to claim 4, wherein a concentration of the 3-HPMA in the urine specimen obtained from the subject to be tested is corrected with a concentration of creatine in the urine specimen.

11. An assessment method according to claim 2, wherein the cerebral infarction comprises asymptomatic cerebral infarction.

12. An assessment method according to claim 2, further comprising using, as an indicator, that a content of acrolein in a blood specimen obtained from the subject to be tested is high as compared to that of the healthy subject.

13. An assessment method according to claim 2, wherein a concentration of the 3-HPMA in the urine specimen obtained from the subject to be tested is corrected with a concentration of creatine in the urine specimen.

14. An assessment method according to claim 11, further comprising using, as an indicator, that a content of acrolein in a blood specimen obtained from the subject to be tested is high as compared to that of the healthy subject.

15. An assessment method according to claim 11, wherein a concentration of the 3-HPMA in the urine specimen obtained from the subject to be tested is corrected with a concentration of creatine in the urine specimen.

16. An assessment method according to claim 12, wherein a concentration of the 3-HPMA in the urine specimen obtained from the subject to be tested is corrected with a concentration of creatine in the urine specimen.
